(19) United States
(12) Patent Application Publication
(10) Pub. No.: US 2018/0344644 A1
(43) Pub. Date: Dec. 6, 2018

(54) IMPROVED STABILITY OF LIPOSOME FORMULATIONS AND USES THEREOF

(71) Applicant: Mallinckrodt LLC, Hazelwood, MO (US)

(72) Inventors: Satheesh Podaralla, Creve Coeur, MO (US); Tsz Chung Lai, Manchester, MO (US); Baohua Yue, Ballwin, MO (US)

(21) Appl. No.: 15/761,676

(22) PCT Filed: Sep. 21, 2016
(86) PCT No.: PCT/US2016/052932

§ 371 Date: Mar. 20, 2018

Related U.S. Application Data
(60) Provisional application No. 62/221,244, filed on Sep. 21, 2015.

(51) Int. Cl. A61K 9/127; A61K 31/337; A61K 31/496; A61K 9/00; A61P 35/02

(52) U.S. Cl. CPC A61K 9/1272; A61K 9/1278; A61P 35/02; A61K 31/496; A61K 9/0019; A61K 31/337

(57) ABSTRACT

The present invention provides improved stable liposomal formulations and method of preparation thereof. The liposomal formulations include liposomes containing a phosphatidylcholine lipid, a sterol, a PEG-lipid, and a taxane, wherein the taxane is docetaxel or a derivative thereof. The liposomal formulations have improved stability and extended shelf-life. The present invention also provides pharmaceutical compositions comprising a liposomal formulation for the treatment of cancer.

### CLAIM OF PRIORITY

This application claims priority to U.S. Provisional Application No. 62/221,244, filed on Sep. 21, 2015, which is incorporated herein by reference in its entirety to the full extent permitted by law.

### BACKGROUND OF THE INVENTION

Taxotere® (docetaxel) and Taxol® (paclitaxel) are the most widely prescribed anticancer drugs on the market, and are associated with a number of pharmacological and toxicological concerns, including highly variable (docetaxel) and non-linear (paclitaxel) pharmacokinetics (PK), serious hypersensitivity reactions associated with the formulation vehicle (Cremophor EL, Tween 80), and acute and dose-limited toxicities, such as myelosuppression, neurotoxicity, fluid retention, asthenia, hyperlacrimation, oncholysis and alopecia. In the case of Taxotere, the large variability in PK causes significant variability in toxicity and efficacy, as well as hematological toxicity correlated with systemic exposure to the drug. In addition, since the therapeutic activity of taxanes increases with the duration of tumor cell drug exposure, the dose-limiting toxicity of commercial taxane formulations substantially limits their therapeutic potential. Moreover, drug resistance due to, for example, up-regulation of protein transporter pumps by cancer cells, can further complicate taxane-based therapies.

Docetaxel derivatives have been developed in an effort to overcome these pharmacological and toxicological limitations. For example, U.S. Pat. No. 8,324,410 describes, inter alia, docetaxel derivatives and liposomal compositions thereof to improve the circulation half-life and efficacy, and reduce toxicity. Similarly, paclitaxel liposomal formulations have been found to significantly increase the maximum tolerated dose (MTD) of paclitaxel (e.g., Koudelka et al, J. Control Release, 163(3):322-334 (2012)). However, much of the improvements are dependent on the stable association of the taxane or derivative thereof with the liposome. Indeed, stable long circulating liposomal formulation can increase the amount and exposure of the drug that reaches therapeutic targets thereby improving efficacy, and at the same time, reduce accumulation in healthy tissue thereby reducing toxicity.

One factor limiting the stability of liposomal formulations is the degree of lipid hydrolysis of the lipid membrane. For example, hydrolysis of 1,2-distearoyl-sn-glycero-3-phosphocholine (distearoylphosphatidylcholine; DSPC) leads to the formation of lysophosphatidylcholine (lyso-PC; LSPC), a key degradant, and stearic acid. LSPC has detergent-like properties, which allow it to move back and forth across the liposomal membrane. The presence of LSPC destabilizes the lipid bilayer and increases the permeability of the liposome and, in turn, the potential for dose dumping. Thus, it is essential to minimize the level of LSPC if the liposomal formulation is intended for intravenous administration. From the perspective of product quality and safety, it is critical to keep the levels of LSPC to a minimum in liposomal formulations to prevent leakage of the loaded therapeutic agent during storage and subsequent administration in vivo. In sum, prevention or minimization of hydrolysis of DSPC and maintenance of low levels of LSPC in the liposomal formulations is critical for physical storage and in vivo performance of the liposomal formulations.

In the prior art, the rate of lipid hydrolysis and formation of LSPC was minimized by manipulating the storage temperature, buffer and pH. More specifically, the rate of lipid hydrolysis was found to be minimal at pH 6.5 when stored in a refrigerated condition at 2-8° C. However, liposomal formulations at a pH of 6.5 and storage in a refrigerated condition are not a universal solution for the minimization of lipid hydrolysis. For example, a pH of 6.5 is not optimal of intravenous administration, or for therapeutic agents that are acid-labile.

Accordingly, there in a need in the art for improved stable liposomal formulations. It is essential to develop a stable liposomal formulations with low levels of LSPC for safe and effective delivery of therapeutics. Known literature approaches for reducing the hydrolysis of lipids are not a viable solutions. The present invention addresses this and other needs.

### BRIEF SUMMARY OF THE INVENTION

In one embodiment, the present invention provides liposomal formulations with improved stability. In another embodiment, the present invention provides improved stability of a docetaxel derivative liposomal formulation. In a further embodiment, the present invention provides a liposomal formulation comprising (i) a phosphatidylcholine lipid; (ii) a sterol; (iii) a PEG-lipid; and (iv) a docetaxel derivative, wherein the docetaxel is esterified at the 2′-O-position with a heterocyclyl-(C₂₋₅alkanoic acid).

In another embodiment, the present invention provides liposomal formulations with extended shelf-life. In one embodiment, the liposomal formulation comprises (i) a phosphatidylcholine lipid; (ii) a sterol; (iii) a PEG-lipid; and (iv) a docetaxel derivative, wherein the docetaxel is esterified at the 2′-O-position with a heterocyclyl-(C₂₋₅alkanoic acid).

In a further embodiment, the present invention provides a method of reducing lipid hydrolysis of a liposomal formulation, including docetaxel derivative liposomal formulations. The method includes but is not limited to: (i) adjusting the pH of the liposomal formulation to about 7.1 to about 7.4; (ii) maintaining an intravesicular pH of the liposome between about 5.5 to about 5.8; and/or (iii) employing an ammonium sulfate buffer at concentrations between about 325 mM to about 350 mM.

In yet another embodiment, the present invention provides a pharmaceutical composition comprising a docetaxel derivative liposomal formulation provided herein and a pharmaceutically-acceptable excipient, wherein the liposomal formulation has a pH of about 7.1 to about 7.4. In further embodiment, the docetaxel derivative liposomal formulation has a pH of about 7.3.

In still another embodiment, the present invention provides a method for the treatment of cancer. In a one embodiment, the invention provides a method for treating cancer by administering to a patient in need thereof the pharmaceutical composition of the invention.

### BRIEF DESCRIPTION OF THE DRAWINGS

FIG. 1 shows the percent hydrolysis of DPSC in a liposomal formulation over time at (A) 25° C. and (B) 5° C.

FIG. 2 shows the percent of LSPC/Total PC ratio in a liposomal formulation over time at (A) 25° C. and (B) 5° C.

FIG. 3 shows the percent lipid hydrolysis over the liposomal intravesicular pH in a liposomal formulation: (A) DSPC and (B) LSPC.

FIG. 4 shows contour plots of percent lipid hydrolysis over the ammonium sulfate concentration and external buffer pH: (A) DPSC, (B) LSPC, and (C) Stearic Acid.

### DETAILED DESCRIPTION OF THE INVENTION

### I. General

The present invention provides liposomal formulations having improved stability and extended shelf-life, and methods of reducing lipid hydrolysis of a liposome. The liposomal formulations comprise, for example, a docetaxel derivative, and are useful for the treatment of cancer as described herein.

### II. Definitions

As used herein, the term “liposome” encompasses any compartment enclosed by a lipid bilayer. The term liposome includes unilamellar vesicles which are comprised of a single lipid bilayer and generally have a diameter in the range of about 20 to about 400 nm. Liposomes can also be multilamellar, which generally have a diameter in the range of 1 to 10 μm. In some embodiments, liposomes can include multilamellar vesicles (MLVs; from about 1 μm to about 10 μm in size), large unilamellar vesicles (LUVs; from a few hundred nanometers to about 10 μm in size), and small unilamellar vesicles (SUVs; from about 20 nm to about 200 nm in size).

As used herein, the term “phosphatidylcholine lipid” refers to a diacylglyceride phospholipid having a choline headgroup (i.e., a 1,2-diacyl-sn-glycero-3-phosphocholine). The acyl groups in a phosphatidylcholine lipid are generally derived from fatty acids having from 6 to 24 carbon atoms. Phosphatidylcholine lipids can include synthetic and naturally-derived 1,2-diacyl-sn-glycero-3-phosphocholines.

As used herein, the term “sterol” refers to a steroid containing at least one hydroxyl group. A steroid is characterized by the presence of a fused, tetracyclic gonane ring system. Sterols include, but are not limited to, cholesterol (i.e., 2,15-dimethyl-14-(1,5-dimethylhexyl)tetracyclo[8.7.0.0^{2,7}.0^{11,15}]heptacos-7-en-5-ol; Chemical Abstracts Services Registry No. 57-88-5).

As used herein, the term “PEG-lipid” refers to a poly(ethylene glycol) polymer covalently bound to a hydrophobic or amphipilic lipid moiety. The lipid moiety can include fats, waxes, steroids, fat-soluble vitamins, monoglycerides, diglycerides, phospholipids and sphingolipids. Preferred PEG-lipids include diacyl-phosphatidylethanolamine-N-[methoxy(polyethene glycol)]s and N-acyl-sphingosine-1-{succinyl[methoxy(polyethylene glycol)]}s. The molecular weight of the PEG in the PEG-lipid is generally from about 500 to about 5000 Daltons (Da; g/mol). The PEG in the PEG-lipid can have a linear or branched structure.

As used herein, the term “heterocyclyl” refers to a saturated or unsaturated ring system having from 3 to 12 ring members and from 1 to 4 heteroatoms of N, O and S. The heteroatoms can also be oxidized, such as, but not limited to, —S(O)— and —S(O)₂—. Heterocyclyl groups can include any number of ring atoms, such as, 3 to 6, 4 to 6, 5 to 6, 3 to 8, 4 to 8, 5 to 8, 6 to 8, 3 to 9, 3 to 10, 3 to 11 or 3 to 12 ring members. Any suitable number of heteroatoms can be included in the heterocyclyl groups, such as 1, 2, 3 or 4, or 1 to 2, 1 to 3, 1 to 4, 2 to 3, 2 to 4 or 3 to 4. Heterocyclyl includes, but is not limited to, 4-methylpiperazinyl, morpholino and piperidinyl.

As used herein the term “alkanoic acid” refers to a carboxylic acid containing 2 to 5 carbon atoms. The alkanoic acids may be linear or branched. Examples of alkanoic acids include, but are not limited to, acetic acid, propionic acid and butanoic acid.

As used herein, the terms “molar percentage” and “mol %” refer to the number of a moles of a given lipid component of a liposome divided by the total number of moles of all lipid components. Unless explicitly stated, the amounts of active agents, diluents or other components are not included when calculating the mol % for a lipid component of a liposome.

As used herein, the term “loading” refers to effecting the accumulation of a therapeutic agent, e.g., a docetaxel derivative, in a liposome. The docetaxel derivative can be encapsulated in the aqueous interior of the liposome, or it can be embedded in the lipid bilayer. Liposomes can be passively loaded, wherein the docetaxel derivative is included in the solutions used during liposome preparation. Alternatively, liposomes can be remotely loaded by establishing a chemical gradient (e.g., a pH or ion gradient) across the liposome bilayer, causing migration of the docetaxel derivative from the aqueous exterior to the liposome interior environment or space.

As used herein, the term “insertion” refers to the embedding of a lipid component into a liposome bilayer. In general, an amphiphilic lipid such as a PEG-lipid is transferred from solution to the bilayer due to van der Waals interactions between the hydrophobic portion of the amphiphilic lipid and the hydrophobic interior of the bilayer.

As used herein, the term “composition” refers to a product comprising the specified ingredients in the specified amounts, as well as any product which results, directly or indirectly, from combination of the specified ingredients in the specified amounts. Pharmaceutical compositions of the present invention generally contain a liposome as described herein and a pharmaceutically-acceptable carrier, diluent or excipient. By “pharmaceutically acceptable,” it is meant that the carrier, diluent or excipient must be compatible with the other ingredients of the formulation and non-deleterious to the recipient thereof.

As used herein, the term “cancer” refers to conditions including human cancers and carcinomas, sarcomas, adenocarcinomas, lymphomas, leukemias and solid and lymphoid cancers. Examples of different types of cancer include, but are not limited to, lung cancer (e.g., non-small cell lung cancer or NSCLC), ovarian cancer, prostate cancer, colorectal cancer, liver cancer (i.e., hepatocarcinoma), renal cancer (i.e., renal cell carcinoma), bladder cancer, breast cancer, thyroid cancer, pleural cancer, pancreatic cancer, uterine cancer, cervical cancer, testicular cancer, anal cancer, pancreatic cancer, bile duct cancer, gastrointestinal carcinoid tumors, esophageal cancer, gall bladder cancer, appendix cancer, small intestine cancer, stomach (gastric) cancer, cancer of the central nervous system, cancer of unknown primary origin, skin cancer, choriocarcinoma, head and neck cancer, blood cancer, osteogenic sarcoma, fibrosarcoma, neuroblastoma, glioma, melanoma, B-cell lymphoma, non-Hodgkin's lymphoma, Burkitt's lymphoma, Small Cell lymphoma, Large Cell lymphoma, monocytic leukemia, myelogenous leukemia, acute lymphocytic leukemia, acute myelocytic leukemia and multiple myeloma.

As used herein, the terms “treat”, “treating” and “treatment” refer to any indicia of success in the treatment or amelioration of a cancer or a symptom of cancer, including any objective or subjective parameter such as abatement; remission (e.g., full or partial); achieving a complete response in a patient; achieving a partial response in a patient; maintaining a stable disease state (e.g., the target lesions have not decreased in size, however, the target lesions have also not increased in size and/or new lesions have not formed); diminishing of symptoms or making the cancer or cancer symptom more tolerable to the patient (clinical benefit). The treatment or amelioration of symptoms can be based on any objective or subjective parameter, including, e.g., the result of a physical examination (clinical benefit) or clinical test.

As used herein, the terms “administer”, “administered” or “administering” refer to methods of administering the liposome compositions of the present invention. The liposome compositions of the present invention can be administered in a variety of ways, including parenterally, intravenously, intradermally, intramuscularly or intraperitoneally. The liposome compositions can also be administered as part of a composition or formulation.

As used herein, the term “subject” refers to any mammal, in particular a human, at any stage of life.

The use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Consequently, recitation of ranges of values herein are merely intended to serve as a shorthand method of referring individually to each separate value falling within the range, unless otherwise indicated herein, and each separate value is incorporated into the specification as if it were individually recited herein.

### III. Embodiments of the Invention

In one embodiment, the present invention provides a lipsomal formulation having improved stability. In another embodiment, the lipsomal formulation has extended shelf-life. In one embodiment, the liposomal formulation comprises a phosphatidylcholine lipid, a sterol, a PEG-lipid, and a docetaxel derivative. In another embodiments, the docextael is derivatized at the 2′—OH group with a heterocyclyl-(C₂₋₅alkanoic acid).

In another embodiment, the present invention provides a pharmaceutical composition comprising the liposomal formulation and a pharmaceutically-acceptable excipient.

In still another embodiment, the invention provides a method of reducing lipid hydrolysis of a lipsomal formulation. The method includes but is not limited to: (i) adjusting the pH of the liposomal formulation to about 7.1 to about 7.4; (ii) maintaining an intravesicular pH of the liposome between about 5.5 to about 5.8; and/or (iii) employing an ammonium sulfate buffer at concentrations between about 325 mM to about 350 mM.

In another embodiment, the invention provides liposomal formulation for the treatment of cancer. In a further embodiment, the invention provides a method of treating cancer in a patient in need thereof by administer to the patient a lipsomal formulation of the present invention. In still a further embodiment, the invention provides a method of treating cancer in a patient in need thereof by administer to the patient a lipsomal formulation of the present invention, wherein the liposomal formulation has a pH of about 7.1 to about 7.4, and in particular, a pH of about 7.3.

A. Liposomes

The liposomes of the present invention can contain any suitable lipid, including cationic lipids, zwitterionic lipids, neutral lipids, or anionic lipids as described above. Suitable lipids can include fats, waxes, steroids, cholesterol, fat-soluble vitamins, monoglycerides, diglycerides, phospholipids, sphingolipids, glycolipids, cationic or anionic lipids, derivatized lipids and the like.

In general, the liposomes of the present invention contain at least one phosphatidylcholine (PC) lipid. Suitable PC lipids include saturated PCs and unsaturated PCs.

Examples of saturated PCs include 1,2-dilauroyl-sn-glycero-3-phosphocholine (DLPC), 1,2-dimyristoyl-sn-glycero-3-phosphocholine (dimyristoylphosphatidyl choline; DMPC), 1,2-distearoyl-sn-glycero-3-phosphocholine (distearoylphosphatidylcholine; DSPC), 1,2-dioleoyl-sn-glycero-3-phosphocholine (DOPC), 1,2-dipalmitoyl-sn-glycero-3-phosphocholine (dipalmitoylphosphatidylcholine; DPPC), 1-myristoyl-2-palmitoyl-sn-glycero-3-phosphocholine (MPPC), 1-palmitoyl-2-myristoyl-sn-glycero-3-phosphocholine (PMPC), 1-myristoyl-2-stearoyl-sn-glycero-3-phosphocholine (MSPC), 1-palmitoyl-2-stearoyl-sn-glycero-3-phosphocholine (PSPC), 1-stearoyl-2-palmitoyl-sn-glycero-3-phosphocholine (SPPC) and 1-stearoyl-2-myristoyl-sn-glycero-3-phosphocholine (SMPC).

Examples of unsaturated PCs include, but are not limited to, 1,2-dimyristoleoyl-sn-glycero-3-phosphocholine, 1,2-dimyristelaidoyl-sn-glycero-3-phosphocholine, 1,2-dipamiltoleoyl-sn-glycero-3-phosphocholine, 1,2-dipalmitelaidoyl-sn-glycero-3-phosphocholine, 1,2-dioleoyl-sn-glycero-3-phosphocholine (DOPC), 1,2-dielaidoyl-sn-glycero-3-phosphocholine, 1,2-dipetroselenoyl-sn-glycero-3-phosphocholine, 1,2-dilinoleoyl-sn-glycero-3-phosphocholine, 1-palmitoyl-2-oleoyl-sn-glycero-3-phosphocholine (palmitoyloleoylphosphatidylcholine; POPC), 1-palmitoyl-2-linoleoyl-sn-glycero-3-phosphocholine, 1-stearoyl-2-oleoyl-sn-glycero-3-phosphocholine (SOPC), 1-stearoyl-2-linoleoyl-sn-glycero-3-phosphocholine, 1-oleoyl-2-myristoyl-sn-glycero-3-phosphocholine (OMPC), 1-oleoyl-2-palmitoyl-sn-glycero-3-phosphocholine (OPPC) and 1-oleoyl-2-stearoyl-sn-glycero-3-phosphocholine (DSPC).

Lipid extracts, such as egg PC, heart extract, brain extract, liver extract, soy PC and hydrogenated soy PC (HSPC) are also useful in the present invention.

The liposomal formulations provided herein will, in some embodiments, consist essentially of PC/cholesterol mixtures (with an added therapeutic agent and PEG-lipid as described below). In some embodiments, the liposomal formulations will consist essentially of a phosphatidylcholine lipid or mixture of phosphatidylcholine lipids, with cholesterol, a PEG-lipid and a therapeutic agent. In still other embodiments, the liposomal formulations will consist essentially of a single type of phosphatidylcholine lipid, with cholesterol, a PEG-lipid and a therapeutic agent. In some embodiments, when a single type of phosphatidylcholine lipid is used, it is selected from the group consisting of DOPC, DSPC, HSPC, DPPC, POPC and SOPC.

In some embodiments, the phosphatidylcholine lipid is selected from the group consisting of DPPC, DSPC, HSPC and mixtures thereof. In some embodiments, the liposomal formulations of the present invention include liposomes containing about 50 to about 65 mol % of a phosphatidylcholine lipid or mixture of phosphatidylcholine lipids, about 53 to about 56 mol % of a phosphatidylcholine lipid or mixture of phosphatidylcholine lipids, about 50 to about 56 mol % of a phosphatidylcholine lipid or mixture of phosphatidylcholine lipids, or about 45 to about 70 mol % of a phosphatidylcholine lipid or mixture of phosphatidylcholine lipids. The liposomes can contain, for example, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69 or about 70 mol % a phosphatidylcholine lipid or a mixture thereof. In some embodiments, the liposomes contain about 56 mol % a phosphatidylcholine lipid or a mixture thereof. In still other embodiments, the liposomes contain about 55 mol % a phosphatidylcholine lipid or a mixture thereof. In additional embodiments, the liposomes contain about 54 mol % a phosphatidylcholine lipid or a mixture thereof. In further embodiments, the liposomes contain about 53 mol % a phosphatidylcholine lipid or a mixture thereof.

The liposomes can contain, for example, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69 or about 70 mol % phosphatidylcholine. In some embodiments, the liposomes contain about 56 mol % phosphatidylcholine. In still other embodiments, the liposomes contain about 55 mol % phosphatidylcholine. In additional embodiments, the liposomes contain about 54 mol % phosphatidylcholine. In further embodiments, the liposomes contain about 53 mol % phosphatidylcholine.

Other suitable phospholipids, generally used in low amounts or in amounts less than the phosphatidylcholine lipids, include phosphatidic acids (PAs), phosphatidylethanolamines (PEs), phosphatidylglycerols (PGs), phosphatidylserine (PSs) and phosphatidylinositol (PIs). Examples of phospholipids include, but are not limited to, dimyristoylphosphatidylglycerol (DMPG), distearoylphosphatidylglycerol (DSPG), dioleoylphosphatidylglycerol (DOPG), dipalmitoylphosphatidylglycerol (DPPG), dimyristoylphosphatidylserine (DMPS), distearoylphosphatidyl serine (DSPS), dioleoylphosphatidylserine (DOPS), dipalmitoylphosphatidylserine (DPPS), dioleoylphosphatidylethanolamine (DOPE), palmitoyloleoylphosphatidylcholine (POPC), palmitoyloleoylphosphatidylethanolamine (POPE), dipalmitoylphosphatidylethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearoyl-2-oleoyl-phosphatidyethanol amine (SOPE), dielaidoylphosphoethanolamine (transDOPE) and cardiolipin.

In some embodiments, phospholipids can include reactive functional groups for further derivatization. Examples of such reactive lipids include, but are not limited to, dioleoylphosphatidylethanolamine-4-(N-maleimidomethyl)-cyclohexane-1-carboxylate (DOPE-mal) and dipalmitoylphosphatidylethanolamine-N-succinyl (succinyl-PE).

Liposomes of the present invention can contain steroids, characterized by the presence of a fused, tetracyclic gonane ring system. Examples of steroids include, but are not limited to, cholic acid, progesterone, cortisone, aldosterone, testosterone, dehydroepiandrosterone and sterols, such as estradiol and cholesterol. Synthetic steroids and derivatives thereof are also contemplated for use in the present invention.

In general, the liposomes contain at least one sterol. In some embodiments, the sterol is cholesterol (i.e., 2,15-dimethyl-14-(1,5-dimethylhexyl)tetracyclo[8.7.0.0^{2,7}.0^{11,15}]heptacos-7-en-5-ol). In some embodiments, the liposomes can contain about 30 to about 50 mol % of cholesterol or about 30 to about 45 mol % of cholesterol. The liposomes can contain, for example, about 30, about 31, about 32, about 33, about 34, about 35, about 36, about 37, about 38, about 39, about 40, about 41, about 42, about 43, about 44, about 45, about 46, about 47, about 48, about 49 or about 50 mol % cholesterol. In some embodiments, the liposomes contain about 30 to about 40 mol % cholesterol. In some embodiments, the liposomes contain about 40 to about 45 mol % cholesterol. In additional embodiments, the liposomes contain about 40 to about 50 mol % cholesterol. In some embodiments, the liposomes contain about 45 mol % cholesterol. In some embodiments, the liposomes contain about 44 mol % cholesterol.

The liposomes of the present invention can include any suitable poly(ethylene glycol)-lipid derivative (PEG-lipid). In some embodiments, the PEG-lipid is a diacyl-phosphatidylethanolamine-N-[methoxy(polyethene glycol)]. The molecular weight of the poly(ethylene glycol) in the PEG-lipid is generally in the range of from about 500 Da to about 5000 Da. The poly(ethylene glycol) can have a molecular weight of, for example, 750 Da, 1000 Da, 2000 Da, or 5000 Da. In some embodiments, the PEG-lipid is selected from distearoyl-phosphatidylethanolamine-N-[methoxy(polyethene glycol)-2000] (DSPE-PEG-2000) and distearoyl-phosphatidylethanolamine-N-[methoxy(polyethene glycol)-5000] (DSPE-PEG-5000). In some embodiments, the PEG-lipid is DSPE-PEG-2000.

In general, the liposomal formulations of the present invention include liposomes containing about 2 to about 8 mol % of the PEG-lipid or about 1 to about 10 mol % of the PEG-lipid. The liposomes can contain, for example, about 1, about 2, about 3, about 4, about 5, about 6, about 7, about 8, about 9 or about 10 mol % PEG-lipid. In some embodiments, the liposomes contain 2 to 6 mol % PEG-lipid. In some embodiments, the liposomes contain about 3 mol % PEG-lipid. In some embodiments, the liposomes contain about 3 mol % DSPE-PEG-2000.

The liposomes of the present invention can also include some amounts of cationic lipids, which are generally amounts lower than the amount of phosphatidylcholine lipid. Cationic lipids contain positively charged functional groups under physiological conditions. Cationic lipids include, but are not limited to, N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N-[1-(2,3,-ditetradecyloxy)propyl]-N,N-dimethyl-N-hydroxyethylammonium bromide (DMRIE), N-[1-(2,3,dioleyloxy)propyl]-N,N-dimethyl-N-hydroxy ethylammonium bromide (DORIE), 3β-[N—(N′,N′-dimethylaminoethane) carbamoyl]cholesterol (DC-Chol), dimethyldioctadecylammonium (DDAB) and N,N-dimethyl-2,3-dioleyloxy)propyl amine (DODMA).

In some embodiments of the present invention, the liposome includes from about 50 mol % to about 70 mol % of DSPC and from about 25 mol % to about 45 mol % of cholesterol. In some embodiments, the liposome includes about 53 mol % of DSPC, about 44 mol % of cholesterol and about 3 mol % of DSPE-PEG-2000. In some embodiments, the liposome includes about 66 mol % of DSPC, about 30 mol % of cholesterol and about 4 mol % of DSPE-PEG-2000.

B. Methods for Preparing Stable Liposomal Formulations

Liposomes can be prepared and loaded with a therapeutic agent using a number of techniques that are known to those of skill in the art. The invention provides methods for preparing a stable liposomal formulation. In one embodiment, a method for preparing a stable liposomal formulation includes the steps of: (a) forming a first liposome having a lipid bilayer including a phosphatidylcholine lipid and a sterol, wherein the lipid bilayer encapsulates an interior environment or space containing an aqueous solution; (b) loading the first liposome with a therapeutic agent, or a pharmaceutically-acceptable salt thereof, to form a loaded liposome; (c) incorporating the PEG-lipid into the lipid bilayer of the loaded liposome; and (d) adjusting the pH to about 7.1 to about 7.4. In a further embodiment, in step (d), the pH is adjusted to about 7.3. In some embodiments, the therapeutic agent is docetaxel esterified at the 2′-O-position with a heterocyclyl-(C₂₋₅alkanoyl) group.

In another embodiment, a method for preparing a stable liposomal formulation includes the steps of: (a) forming a first liposome having a lipid bilayer including a phosphatidylcholine lipid and a sterol, wherein the lipid bilayer encapsulates an interior environment or space containing an aqueous solution; (b) incorporating the PEG-lipid into the lipid bilayer of the first liposome; (c) loading the first liposome with a therapeutic agent, or a pharmaceutically-acceptable salt thereof, to form a loaded liposome; and (d) adjusting the pH to about 7.1 to about 7.4. In a further embodiment, in step (d), the pH is adjusted to about 7.3. In some embodiments, the therapeutic agent is docetaxel esterified at the 2′-O-position with a heterocyclyl-(C₂₋₅alkanoyl) group.

In another embodiment, a method for preparing a stable liposomal formulation includes the steps of: (a) forming a liposome having a lipid bilayer including a phosphatidylcholine lipid and a sterol, wherein the lipid bilayer encapsulates an interior environment or space containing an aqueous solution and a therapeutic agent; (b) incorporating the PEG-lipid into the lipid bilayer of the liposome; and (c) adjusting the pH to about 7.1 to about 7.4. In a further embodiment, in step (c), the pH is adjusted to about 7.3. In some embodiments, the therapeutic agent is docetaxel esterified at the 2′-O-position with a heterocyclyl-(C₂₋₅alkanoyl) group.

Lipid vesicles can be prepared, for example, by hydrating a dried lipid film (prepared via evaporation of a mixture of the lipid and an organic solvent in a suitable vessel) with water or an aqueous buffer. Hydration of lipid films typically results in a suspension of multilamellar vesicles (MLVs). Alternatively, MLVs can be formed by diluting a solution of a lipid in a suitable solvent, such as a C₁₋₄ alkanol, with water or an aqueous buffer. Unilamellar vesicles can be formed from MLVs via sonication or extrusion through membranes with defined pore sizes.

Encapsulation of a therapeutic agent can be conducted by including the agent in the aqueous solution used for film hydration or lipid dilution during MLV formation. Therapeutic agents can also be encapsulated in pre-formed vesicles using “remote loading” techniques. Remote loading includes the establishment of a pH- or ion-gradient on either side of the vesicle membrane, which drives the therapeutic agent from the exterior solution to the interior of the vesicle.

In some embodiments, loading conditions generally include forming an ion-gradient across the liposomal membrane. In one embodiment, an ammonium sulfate concentration gradient is formed across the liposomal membrane. In a further embodiment, the interior of the liposome has a higher concentration of ammonium sulfate than in the exterior aqueous solution. The concentration of ammonium sulfate of the exterior solution can range from about 300 mM to about 400 mM, from about 325 mM to about 375 mM or about 325 mM to about 350 mM. The ammonium sulfate concentration can be about 300 mM, about 305 mM, about 310 mM, about 315 mM, about 320 mM, about 325 mM, about 330 mM, about 335 mM, about 340 mM, about 345 mM, about 350 mM, about 355 mM, about 360 mM, about 365 mM, about 370 mM, about 375 mM, about 380 mM, about 385 mM, about 390 mM, about 395 mM or about 400 mM.

In some embodiments, loading conditions generally include forming a pH gradient across the liposomal membrane to produce liposomes with an acidic liposomal interior and an exterior environment with higher pH than the liposome interior environment or space (e.g., neutral pH) (e.g., Maurer, N., Fenske, D., and Cullis, P. R. (2001) Developments in liposomal drug delivery systems. Expert Opinion in Biological Therapy 1, 923-47; Cullis et al., Biochim Biophys Acta., 1331: 187-211 (1997); Fenske et al., Liposomes: A practical approach. Second Edition. V. Torchilin and V. Weissig, eds., Oxford University Press, p. 167-191 (2001)). In some embodiments, the internal pH of the liposome can range from about 5.0 to about 6.0. In additional embodiments, the internal pH of the liposome can range from about 5.2 to about 5.9. In still other embodiments, the internal pH of the liposome can range from about 5.5 to about 5.8. More specifically, the internal pH of the liposome can be about 5.5, about 5.6, about 5.7 or about 5.8.

In some embodiments, the external pH of the liposomal formulation can range from about 7.0 to about 7.5 or about 7.1 to about 7.4. For example, the pH of the liposomal formulation can be about 7.10, about 7.11, about 7.12, about 7.13, about 7.14, about 7.15, about 7.16, about 7.17, about 7.18, about 7.19, about 7.20, about 7.21, about 7.22, about 7.23, about 7.24, about 7.25, about 7.26, about 7.27, about 7.28, about 7.29, about 7.30, about 7.31, about 7.32, about 7.33, about 7.34, about 7.35, about 7.36, about 7.37, about 7.38, about 7.39 or about 7.40. More specifically, the external pH of the liposomal formulation can be about 7.3.

In some embodiments, the loading step is conducted at a temperature above the gel-to-fluid phase transition temperature (Tₘ) of one or more of the lipid components in the liposomes. The loading can be conducted, for example, at about 50, about 55, about 60, about 65, or at about 70° C. In some embodiments, the loading step is conducted at a temperature of from about 50° C. to about 70° C. Loading can be conducted using any suitable amount of the therapeutic agent. In some embodiments, the docetaxel derivative is used in an amount such that the ratio of the combined weight of the phosphatidylcholine and the sterol in the liposome to the weight of the taxane is from about 1:0.01 to about 1:1. The ratio of the combined phosphatidylcholine/sterol to the weight of the taxane can be, for example, about 1:0.01, about 1:0.05, about 1:0.10, about 1:0.15, about 1:0.20, about 1:0.25, about 1:0.30, about 1:0.35, about 1:0.40, about 1:0.45, about 1:0.50, about 1:0.55, about 1:0.60, about 1:0.65, about 1:0.70, about 1:0.75, about 1:0.80, about 1:0.85, about 1:0.90, about 1:0.95 or about 1:1. In some embodiments, the loading step is conducted such that the ratio of the combined weight of the phosphatidylcholine and the sterol to the weight of the docetaxel derivative is from about 1:0.01 to about 1:1. In some embodiments, the ratio of the combined weight of the phosphatidylcholine and the sterol to the weight of the docetaxel derivative is from about 1:0.05 to about 1:0.5. In some embodiments, the ratio of the combined weight of the phosphatidylcholine and the sterol to the weight of the docetaxel derivative is about 1:0.2. The loading step can be conducted for any amount of time that is sufficient to allow accumulation of the docetaxel derivative in the liposome interior environment or space at a desired level.

The PEG-lipid can also be incorporated into lipid vesicles at various stages of the liposome preparation. For example, MLVs containing a PEG-lipid can be prepared prior to loading with a taxane. Alternatively, a PEG-lipid can be inserted into a lipid bilayer after the loading of a vesicle with a taxane. The PEG-lipid can be inserted into MLVs prior to extrusion of SUVs, or the PEG-lipid can be inserted into pre-formed SUVs.

In some embodiments, the insertion of the PEG-lipid is conducted at a temperature of from about 35° C. to about 70° C. The loading can be conducted, for example, at about 35° C., about 40° C., about 45° C., about 50° C., about 55° C., about 60° C., about 65° C. or at about 70° C. In some embodiments, insertion of the PEG-lipid is conducted at a temperature of from about 50° C. to about 55° C. Insertion can be conducted using any suitable amount of the PEG-lipid. In general, the PEG-lipid is used in an amount such that the ratio of the combined number of moles of the phosphatidylcholine and the sterol to the number of moles of the PEG-lipid is from about 1000:1 to about 20:1. The molar ratio of the combined phosphatidylcholine/sterol to PEG-lipid can be, for example, about 1000:1, about 950:1, about 900:1, about 850:1, about 800:1, about 750:1, about 700:1, about 650:1, about 600:1, about 550:1, about 500:1, about 450:1, about 400:1, about 350:1, about 300:1, about 250:1, about 200:1, about 150:1, about 100:1, about 50:1 or about 20:1. In some embodiments, the loading step is conducted such that the ratio of combined phosphatidylcholine and sterol to PEG-lipid is from about 1000:1 to about 20:1 (mol:mol). In some embodiments, the ratio of the combined phosphatidylcholine and sterol to the PEG-lipid is from about 100:1 to about 20:1 (mol:mol). In some embodiments, the ratio of the combined phosphatidylcholine and sterol to the PEG-lipid is from about 35:1 (mol:mol) to about 25:1 (mol:mol). In some embodiments, the ratio of the combined phosphatidylcholine and sterol to the PEG-lipid is about 33:1 (mol:mol). In some embodiments, the ratio of the combined phosphatidylcholine and sterol to the PEG-lipid is about 27:1 (mol:mol).

A number of additional preparative techniques known to those of skill in the art can be included in the methods of the invention. Liposomes can be exchanged into various buffers by techniques including dialysis, size exclusion chromatography, diafiltration and ultrafiltration. Aqueous buffers and certain organic solvents can be removed from the liposomes via lyophilization.

The present invention provides a method for preparing a stable liposomal formulation that includes the steps of: a) forming a first liposome having a lipid bilayer including a phosphatidylcholine lipid and a sterol, wherein the lipid bilayer encapsulates an interior environment or space containing an aqueous solution; b) loading the first liposome with a therapeutic agent, or a pharmaceutically-acceptable salt thereof, to form a loaded liposome; c) incorporating the PEG-lipid into the lipid bilayer of the loaded liposome; and (d) adjusting the pH to about 7.1 to about 7.4, or about 7.3. In some embodiments, the therapeutic agent is docetaxel esterified at the 2′-O-position with a heterocyclyl-(C₂₋₅alkanoyl) group.

The present invention provides liposomal formulations having extended shelf-life from about 12 to about 36 months or about 18 months to about 30 months. Specifically, the liposomal formulations have an extended shelf-life of about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35 or 36 months. More specifically, the liposomal formulations have an extended shelf-life of about 24 months.

C. Therapeutic Agents

The liposomes of the present invention contain a therapeutic agent. In some embodiments, the therapeutic agent is a taxane. In a further embodiment, the taxane is a docetaxel or a derivative thereof, e.g., according to U.S. Pat. No. 8,324,410, incorporated-by-reference herein. In still a further embodiment, the docetaxel derivative has the following formula:

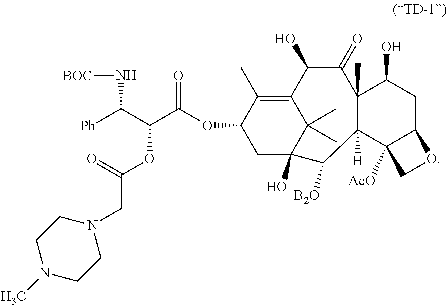

In other embodiment, the taxane derivative is a pharmaceutically-acceptable salt of TD-1.

In another embodiment, suitable therapeutic agents include, but are not limited to, amphotericin B, doxorubicin, bupivacaine camptothecin, carfilzomib, daunorubicin, vincristine, cisplatin, oxaloplatin, irinotecan, topotecan, annamycin, cytarabine, paclitaxel, sunitinib, imatinib, lapatinib, mitomycin C, epirubicin, pirarubicin, rubidomycin, carcinomycin, N-acetyladriamycin, rubidazone, 5-imido daunomycin, N-acetyl daunomycin, daunory line, mitoxanthrone, morphine, camptothecin, 9-aminocamptothecin, 7-ethylcamptothecin, 7-Ethyl-10-hydroxy-camptothecin, 10-hydroxycamptothecin, 9-nitrocamptothecin,1O, 11-methylenedioxycamptothecin, 9-amino-1O, 11-methylenedioxycamptothecin, 9-chloro-10, 11-methylenedioxycamptothecin, irinotecan, lurtotecan, silatecan, (7-(4-methylpiperazinomethylene)-10,11-ethylenedioxy-20(S)-camptothecin, 7-(4-methylpiperazinomethylene)-10, II-methylenedioxy-20(S)-camptothecin, 7-(2-N-isopropylamino)ethyl)-(20S)-camptothecin, CKD-602, vincristine, vinblastine, vinorelbine, vinflunine, vinpocetine, vindesine, verteporfin, ellipticine, 6-3-aminopropyl-ellipticine, 2-diethylaminoethyl-ellipticinium, datelliptium, retelliptine, paclitaxel, docetaxel, diclofenac, bupivacaine, 17-Dimethylaminoethyl amino-17-demethoxygeldanamycin, cetirizine, fexofenadine, primidone and other catecholamines, epinephrine, (S)-2-(2,4-dihydroxyphenyl)-4,5-dihydro-4-methyl-4-thiazolecarboxylic acid (deferitrin), (S)-4,5-dihydro-2-(3-hydroxy-2-pyridinyl)-4-methyl-4-thiazolecarboxylic acid (desferrithiocin), (S)-4,5-dihydro-2-[2-hydroxy-4-(3,6,9,12-tetraoxamidecyloxy)phenyl]-4-methyl-4-thiazolecarboxylic acid, (S)-4,5-dihydro-2-[2-hydroxy-4-(3,6-dioxaheptyloxy)phenyl]-4-methyl-4-thiazolecarboxylic acid, ethyl (S)-4,5-dihydro-2-[2-hydroxy-4-(3,6-dioxaheptyloxy)phenyl]-4-methyl-4-thiazolecarboxylate, (S)-4,5-dihydro-2-[2-hydroxy-3-(3,6,9-trioxadecyloxy)]-4-methyl-4-thiazolecarboxylic acid and desazadesferrithiocin, including salts, prodrugs and derivatives thereof.

In yet another embodiment, the therapeutic agent is an anti-cancer drug, such as those disclosed in US Publication No. 2014/0220111 that is incorporated herein in its entirety.

D. Diagnostic Agents

The liposomes of the present invention may also contain diagnostic agents. A diagnostic agent used in the present invention can include any diagnostic agent known in the art, as provided, for example, in the following references: Armstrong et al., Diagnostic Imaging, 5th Ed., Blackwell Publishing (2004); Torchilin, V. P., Ed., Targeted Delivery of Imaging Agents, CRC Press (1995); Vallabhajosula, S., Molecular Imaging: Radiopharmaceuticals for PET and SPECT, Springer (2009). A diagnostic agent can be detected by a variety of ways, including as an agent providing and/or enhancing a detectable signal that includes, but is not limited to, gamma-emitting, radioactive, echogenic, optical, fluorescent, absorptive, magnetic or tomography signals. Techniques for imaging the diagnostic agent can include, but are not limited to, single photon emission computed tomography (SPECT), magnetic resonance imaging (MRI), optical imaging, positron emission tomography (PET), computed tomography (CT), x-ray imaging, gamma ray imaging and the like. The diagnostic agents can be associated with the therapeutic liposome in a variety of ways, including, for example, being embedded or encapsulated in the liposome.

In some embodiments, a diagnostic agent can include chelators that bind to metal ions to be used for a variety of diagnostic imaging techniques. Exemplary chelators include, but are not limited to, ethylenediaminetetraacetic acid (EDTA), [4-(1,4,8, 11-tetraazacyclotetradec-1-yl) methyl]benzoic acid (CPTA), cyclohexanediaminetetraacetic acid (CDTA), ethylenebis(oxyethylenenitrilo)tetraacetic acid (EGTA), diethylenetriaminepentaacetic acid (DTPA), citric acid, hydroxyethyl ethylenediamine triacetic acid (HEDTA), iminodiacetic acid (IDA), triethylene tetraamine hexaacetic acid (TTHA), 1,4,7, 10-tetraazacyclododecane-1,4,7,10-tetra(m ethylene phosphonic acid) (DOTP), 1,4,8,11-tetraazacyclododecane-1,4,8,11-tetraacetic acid (TETA), 1,4,7,10-tetraazacyclododecane-1,4,7,10-tetraacetic acid (DOTA) and derivatives thereof.

A radioisotope can be incorporated into some of the diagnostic agents described herein and can include radionuclides that emit gamma rays, positrons, beta and alpha particles and X-rays. Suitable radionuclides include but are not limited to ²²⁵Ac, ⁷²As, ²¹¹At, ¹¹B, ¹²⁸Ba, ²¹²Bi, ⁷⁵Br, ⁷⁷Br, ¹⁴C, ¹⁰⁹Cd, ⁶²Cu, ⁶⁴Cu, ⁶⁷Cu, ¹⁸F, ⁶⁷Ga, ⁶⁸Ga ³H, ¹²³I, ¹²⁵I, ¹³⁰I, ¹³¹I, ¹¹¹In, ¹⁷⁷Lu, ¹³N, ¹⁵O, ³²P, ³³P, ²¹²Pb, ¹⁰³Pd, ¹⁸⁶Re, ¹⁸⁸Re, ⁴⁷Sc, ¹⁵³Sm, ⁸⁹Sr, ⁹⁹mTc, ⁸⁸Y and ⁹⁰Y. In certain embodiments, radioactive agents can include ¹¹¹In-DTPA, ^{99m}Tc(CO)₃-DTPA, ^{99m}Tc(CO)₃-ENPy2, ^{62/64/67}Cu-TETA, ^{99m}Tc(CO)₃-IDA and ^{99m}Tc(CO)₃triamines (cyclic or linear). In other embodiments, the agents can include DOTA and its various analogs with ¹¹¹In, ¹⁷⁷Lu, ¹⁵³Sm, ^{88/90}Y, ^{62/64/67}Cu or ^{67/68}Ga. In some embodiments, the liposomes can be radiolabeled, for example, by incorporation of lipids attached to chelates, such as DTPA-lipid, as provided in the following references: Phillips et al., Wiley Interdisciplinary Reviews: Nanomedicine and Nanobiotechnology, 1(1): 69-83 (2008); Torchilin, V. P. & Weissig, V., Eds. Liposomes 2nd Ed.: Oxford Univ. Press (2003); Elbayoumi, T. A. & Torchilin, V. P., Eur. J. Nucl. Med. Mol. Imaging 33:1196-1205 (2006); Mougin-Degraef, M. et al., Int'l J. Pharmaceutics 344:110-117 (2007).

In other embodiments, the diagnostic agents can include optical agents such as fluorescent agents, phosphorescent agents, chemiluminescent agents and the like. Numerous agents (e.g., dyes, probes, labels, or indicators) are known in the art and can be used in the present invention. (See, e.g., Invitrogen, The Handbook-A Guide to Fluorescent Probes and Labeling Technologies, Tenth Edition (2005)). Fluorescent agents can include a variety of organic and/or inorganic small molecules or a variety of fluorescent proteins and derivatives thereof. For example, fluorescent agents can include, but are not limited to, cyanines, phthalocyanines, porphyrins, indocyanines, rhodamines, phenoxazines, phenylxanthenes, phenothiazines, phenoselenazines, fluoresceins, benzoporphyrins, squaraines, dipyrrolo pyrimidones, tetracenes, quinolines, pyrazines, corrins, croconiums, acridones, phenanthridines, rhodamines, acridines, anthraquinones, chalcogenopyrylium analogues, chlorins, naphthalocyanines, methine dyes, indolenium dyes, azo compounds, azulenes, azaazulenes, triphenyl methane dyes, indoles, benzoindoles, indocarbocyanines, benzoindocarbocyanines, and BODIPY™ derivatives having the general structure of 4,4-difluoro-4-bora-3a,4a-diaza-s-indacene and/or conjugates and/or derivatives of any of these. Other agents that can be used include, but are not limited to, for example, fluorescein, fluorescein-polyaspartic acid conjugates, fluorescein-polyglutamic acid conjugates, fluorescein-polyarginine conjugates, indocyanine green, indocyanine-dodecaaspartic acid conjugates, indocyanine-polyaspartic acid conjugates, isosulfan blue, indole disulfonates, benzoindole disulfonate, bis(ethylcarboxymethyl)indocyanine, bis(pentylcarboxymethyl)indocyanine, polyhydroxyindole sulfonates, polyhydroxybenzoindole sulfonate, rigid heteroatomic indole sulfonate, indocyaninebispropanoic acid, indocyaninebishexanoic acid, 3,6-dicyano-2,5-[(N,N,N′,N′-tetrakis(carboxymethyl)amino]pyrazine, 3,6-[(N,N,N′,N′-tetrakis(2-hydroxyethyl)amino]pyrazine-2,5-dicarboxylic acid, 3,6-bis(N-azatedino)pyrazine-2,5-dicarboxylic acid, 3,6-bis(N-morpholino)pyrazine-2,5-dicarboxylic acid, 3,6-bis(N-piperazino)pyrazine-2,5-dicarboxylic acid, 3,6-bis(N-thiomorpholino)pyrazine-2,5-dicarboxylic acid, 3,6-bis(N-thiomorpholino)pyrazine-2,5-dicarboxylic acid S-oxide, 2,5-dicyano-3,6-bis(N-thiomorpholino)pyrazine S,S-dioxide, indocarbocyaninetetrasulfonate, chloroindocarbocyanine and 3,6-diaminopyrazine-2,5-dicarboxylic acid.

One of ordinary skill in the art will appreciate that particular optical agents used can depend on the wavelength used for excitation, depth underneath skin tissue and other factors generally well known in the art. For example, optimal absorption or excitation maxima for the optical agents can vary depending on the agent employed, but in general, the optical agents of the present invention will absorb or be excited by light in the ultraviolet (UV), visible or infrared (IR) range of the electromagnetic spectrum. For imaging, dyes that absorb and emit in the near-IR (˜700-900 nm, e.g., indocyanines) are preferred. For topical visualization using an endoscopic method, any dyes absorbing in the visible range are suitable.

In some embodiments, the non-ionizing radiation employed in the process of the present invention can range in wavelength from about 350 nm to about 1200 nm. In one exemplary embodiment, the fluorescent agent can be excited by light having a wavelength in the blue range of the visible portion of the electromagnetic spectrum (from about 430 nm to about 500 nm) and emits at a wavelength in the green range of the visible portion of the electromagnetic spectrum (from about 520 nm to about 565 nm). For example, fluorescein dyes can be excited with light with a wavelength of about 488 nm and have an emission wavelength of about 520 nm. As another example, 3,6-diaminopyrazine-2,5-dicarboxylic acid can be excited with light having a wavelength of about 470 nm and fluoresces at a wavelength of about 532 nm. In another embodiment, the excitation and emission wavelengths of the optical agent may fall in the near-infrared range of the electromagnetic spectrum. For example, indocyanine dyes, such as indocyanine green, can be excited with light with a wavelength of about 780 nm and have an emission wavelength of about 830 nm.

In yet other embodiments, the diagnostic agents can include, but are not limited to, magnetic resonance (MR) and x-ray contrast agents that are generally well known in the art, including, for example, iodine-based x-ray contrast agents, superparamagnetic iron oxide (SPIO), complexes of gadolinium or manganese and the like. (See, e.g., Armstrong et al., Diagnostic Imaging, 5th Ed., Blackwell Publishing (2004)). In some embodiments, a diagnostic agent can include a MR imaging agent. Exemplary MR agents include, but are not limited to, paramagnetic agents, superparamagnetic agents and the like. Exemplary paramagnetic agents can include, but are not limited to, gadopentetic acid, gadoteric acid, gadodiamide, gadolinium, gadoteridol, mangafodipir, gadoversetamide, ferric ammonium citrate, gadobenic acid, gadobutrol and gadoxetic acid. Superparamagnetic agents can include, but are not limited to, superparamagnetic iron oxide and ferristene. In certain embodiments, the diagnostic agents can include x-ray contrast agents as provided, for example, in the following references: H. S Thomsen, R. N. Muller and R. F. Mattrey, Eds., Trends in Contrast Media, (Berlin: Springer-Verlag, 1999); P. Dawson, D. Cosgrove and R. Grainger, Eds., Textbook of Contrast Media (ISIS Medical Media 1999); Torchilin, V. P., Curr. Pharm. Biotech. 1:183-215 (2000); Bogdanov, A. A. et al., Adv. Drug Del. Rev. 37:279-293 (1999); Sachse, A. et al., Investigative Radiology 32(1):44-50 (1997). Examples of x-ray contrast agents include, without limitation, iopamidol, iomeprol, iohexol, iopentol, iopromide, iosimide, ioversol, iotrolan, iotasul, iodixanol, iodecimol, ioglucamide, ioglunide, iogulamide, iosarcol, ioxilan, iopamiron, metrizamide, iobitridol and iosimenol. In certain embodiments, the x-ray contrast agents can include iopamidol, iomeprol, iopromide, iohexol, iopentol, ioversol, iobitridol, iodixanol, iotrolan and iosimenol.

E. Targeting Agents

In some cases, liposome accumulation at a target site may be due to the enhanced permeability and retention characteristics of certain tissues such as cancer tissues. Accumulation in such a manner often results in part because of liposome size and may not require special targeting functionality. In other cases, the liposomes of the present invention can also include a targeting agent. Generally, the targeting agents of the present invention can associate with any target of interest, such as a target associated with an organ, tissues, cell, extracellular matrix or intracellular region. In certain embodiments, a target can be associated with a particular disease state, such as a cancerous condition. In some embodiments, the targeting component can be specific to only one target, such as a receptor. Suitable targets can include, but are not limited to, a nucleic acid, such as a DNA, RNA, or modified derivatives thereof. Suitable targets can also include, but are not limited to, a protein, such as an extracellular protein, a receptor, a cell surface receptor, a tumor-marker, a transmembrane protein, an enzyme or an antibody. Suitable targets can include a carbohydrate, such as a monosaccharide, disaccharide or polysaccharide that can be, for example, present on the surface of a cell.

In certain embodiments, a targeting agent can include a target ligand (e.g., an RGD-containing peptide), a small molecule mimic of a target ligand (e.g., a peptide mimetic ligand) or an antibody or antibody fragment specific for a particular target. In some embodiments, a targeting agent can further include folic acid derivatives, B-12 derivatives, integrin RGD peptides, NGR derivatives, somatostatin derivatives or peptides that bind to the somatostatin receptor, e.g., octreotide and octreotate, and the like. The targeting agents of the present invention can also include an aptamer. Aptamers can be designed to associate with or bind to a target of interest. Aptamers can be comprised of, for example, DNA, RNA and/or peptides, and certain aspects of aptamers are well known in the art. (See. e.g., Klussman, S., Ed., The Aptamer Handbook, Wiley-VCH (2006); Nissenbaum, E. T., Trends in Biotech, 26(8): 442-449 (2008)).

F. Pharmaceutical Compositions

Pharmaceutical compositions of the present invention generally contain liposomal formulations as described herein and a pharmaceutically acceptable carrier. The term “carrier” refers to a typically inert substance used as a diluent or vehicle for the liposomal formulation. The term also encompasses a typically inert substance that imparts cohesive qualities to the composition. Typically, the physiologically acceptable carriers are present in liquid form. Examples of liquid carriers include, but not limited to, physiological saline, phosphate buffer, normal buffered saline (135-150 mM NaCl), water, buffered water, 0.4% saline, 0.3% glycine, 0.3M sucrose (and other carbohydrates), glycoproteins to provide enhanced stability (e.g., albumin, lipoprotein, globulin, etc.) and the like. Since physiologically acceptable carriers are determined in part by the particular composition being administered as well as by the particular method used to administer the composition, there are a wide variety of suitable formulations of pharmaceutical compositions of the present invention (see, e.g., Remington's Pharmaceutical Sciences, Maak Publishing Company, Philadelphia, Pa., 17th ed. (1985)).

The compositions of the present invention may be sterilized by conventional, well-known sterilization techniques or may be produced under sterile conditions. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents, wetting agents and the like, e.g., sodium acetate, sodium lactate, sodium chloride, potassium chloride, calcium chloride, sorbitan monolaurate and triethanolamine oleate. Sugars can also be included for stabilizing the compositions, such as a stabilizer for lyophilized liposome compositions.

Pharmaceutical compositions suitable for parenteral administration, such as, for example, by intraarticular, intravenous, intramuscular, intratumoral, intradermal, intraperitoneal and subcutaneous routes, include aqueous and non-aqueous, isotonic sterile injection solutions. The injection solutions can contain antioxidants, buffers, bacteriostats and solutes that render the formulation isotonic with the blood of the intended recipient, and aqueous and non-aqueous sterile suspensions that can include suspending agents, solubilizers, thickening agents, stabilizers and preservatives. Injection solutions and suspensions can also be prepared from sterile powders, such as lyophilized liposomes. In the practice of the present invention, compositions can be administered, for example, by intravenous infusion, intraperitoneally, intravesically or intrathecally. Parenteral administration and intravenous administration are preferred methods of administration. The formulations of liposome compositions can be presented in unit-dose or multi-dose sealed containers, such as ampoules and vials.

The pharmaceutical composition is preferably in unit dosage form. In such form the composition is subdivided into unit doses containing appropriate quantities of the active component, e.g., a liposome formulation. The unit dosage form can be a packaged composition, the package containing discrete quantities of the pharmaceutical composition. The composition can, if desired, also contain other compatible therapeutic agents.

G. Methods of Treating Cancer

In another embodiment, the invention provides a method of treating cancer. The method includes administering to a subject in need thereof a pharmaceutical composition containing a liposomal formulation as described above. In one embodiment, the liposomal formulation contains a docetaxel derivative, in particular, a docetaxel derivative esterified at the 2′-O-position with a heterocyclyl-(C₂₋₅alkanoyl) group. In therapeutic use for the treatment of cancer, the liposome compositions of the present invention can be administered such that the initial dosage of the docetaxel derivative ranges from about 0.001 mg/kg to about 1000 mg/kg daily. A daily dose of about 0.01 to about 500 mg/kg, or about 0.1 to about 200 mg/kg, or about 1 to about 100 mg/kg, or about 10 to about 50 mg/kg, or about 10 mg/kg, or about 5 mg/kg, or about 2-5 mg/kg or about 1 mg/kg can be used. Further, a daily dose of about 3, about 6, about 12, about 24, about 48, about 80, about 120, about 160, about 190, about 225, about 270 and about 320 mg/m² can be used.

The dosages may be varied depending upon the requirements of the patient, the type and severity of the cancer being treated and the pharmaceutical composition being employed. For example, dosages can be empirically determined considering the type and stage of cancer diagnosed in a particular patient. The dose administered to a patient should be sufficient to affect a beneficial therapeutic response in the patient over time. The size of the dose will also be determined by the existence, nature and extent of any adverse side-effects that accompany the administration of a particular liposome composition in a particular patient. Determination of the proper dosage for a particular situation is within the skill of the practitioner. Generally, treatment is initiated with smaller dosages which are less than the optimum dose of the liposome composition. Thereafter, the dosage is increased by small increments until the optimum effect under circumstances is reached. For convenience, the total daily dosage may be divided and administered in portions during the day, if desired. The duration of the infusion may be extended and/or the infusion may be interrupted in the case of an adverse event, but the total duration of the infusion cannot exceed 2 hours and cannot be resumed for several hours following the initiation of the infusion.

The methods described herein apply especially to solid tumor cancers (solid tumors), which are cancers of organs and tissue (as opposed to hematological malignancies), and ideally epithelial cancers. Examples of solid tumor cancers include bladder cancer, breast cancer, cervical cancer, colorectal cancer (CRC), esophageal cancer, gastric cancer, head and neck cancer, hepatocellular cancer, lung cancer, melanoma, neuroendocrine cancer, ovarian cancer, pancreatic cancer, prostate cancer, renal cancer and thymus cancer. In one group of embodiments, the solid tumor cancer suitable for treatment according to the methods of the invention are selected from CRC, breast cancer and prostate cancer. In another group of embodiments, the methods of the invention apply to treatment of hematological malignancies, including for example multiple myeloma, T-cell lymphoma, B-cell lymphoma, Hodgkin's disease, non-Hodgkins lymphoma, acute myeloid leukemia and chronic myelogenous leukemia.

The pharmaceutical compositions may be administered alone in the methods of the invention, or in combination with other therapeutic agents. The additional agents can be anticancer agents belonging to several classes of drugs such as, but not limited to, cytotoxic agents, VEGF-inhibitors, tyrosine kinase inhibitors, monoclonal antibodies and immunotherapies. Examples of such agents include, but are not limited to, doxorubicin, cisplatin, oxaliplatin, carboplatin, 5-fluorouracil, gemcitabine (anti-metabolite), ramucirumab (VEGF 2 inhibitor), bevacizumab (VEGF inhibitor), trastuzumab (monoclonal antibody HER2 inhibitor), afatinib (EGFR tyrosine kinase inhibitor) and others. Additional anti-cancer agents can include, but are not limited to, 20-epi-1,25 dihydroxyvitamin D3,4-ipomeanol, 5-ethynyluracil, 9-dihydrotaxol, abiraterone, acivicin, aclarubicin, acodazole hydrochloride, acronine, acylfulvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizing morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisaziridinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cactinomycin, calcipotriol, calphostin C, calusterone, camptothecin derivatives, canarypox IL-2, capecitabine, caracemide, carbetimer, carboplatin, carboxamide-amino-triazole, carboxyamidotriazole, carest M3, carmustine, cam 700, cartilage derived inhibitor, carubicin hydrochloride, carzelesin, casein kinase inhibitors, castanospermine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethylnorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocarmycin SA, ebselen, ecomustine, edatrexate, edelfosine, edrecolomab, eflomithine, eflomithine hydrochloride, elemene, elsamitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, fluorocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ilmofosine, ilomastat, imidazoacridones, imiquimod, immunostimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-M, interferons, interleukins, iobenguane, iododoxorubicin, iproplatin, irinotecan, irinotecan hydrochloride, iroplact, irsogladine, isobengazole, isohomohalicondrin B, itasetron, jasplakinolide, kahalalide F, lamellarin-N triacetate, lanreotide, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide 7, lobaplatin, lombricine, lometrexol, lometrexol sodium, lomustine, lonidamine, losoxantrone, losoxantrone hydrochloride, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C inhibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, 06-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum complex, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazofurin, pyrazoloacridine, pyridoxylated hemoglobin polyoxyethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RII retinamide, RNAi, rogletimide, rohitukine, romurtide, roquinimex, rubiginone B1, ruboxyl, safingol, safingol hydrochloride, saintopin, sarcnu, sarcophytol A, sargramostim, SDI 1 mimetics, semustine, senescence derived inhibitor 1, sense oligonucleotides, signal transduction inhibitors, signal transduction modulators, simtrazene, single chain antigen binding protein, sizofuran, sobuzoxane, sodium borocaptate, sodium phenylacetate, solverol, somatomedin binding protein, sonermin, sparfosate sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine, vinorelbine tartrate, vinrosidine sulfate, vinxaltine, vinzolidine sulfate, vitaxin, vorozole, zanoterone, zeniplatin, zilascorb, zinostatin, zinostatin stimalamer and zorubicin hydrochloride.

### IV. Examples

### Example 1. Stability of Liposomal Formulations

The stability of liposomal formulations of the present invention were determined in an aqueous buffer at different temperatures, pH and ammonium sulfate concentration (mM). Liposomal formulations containing docetaxel derivative, TD-1, were evaluated for DSPC hydrolysis and formation of LSPC and stearic acid. Table 1 below provides the composition of the liposomal formulations.

TABLE 1
Composition of the Liposomal Formulations

| | | | DSPE- | | External |
|---|---|---|---|---|---|
| Sample | DSPC | Cholesterol | PEG2000 | TD1 | buffer |
| No | (mg/mL) | (mg/mL) | (mg/mL) | (mg/mL) | pH |
| 1 | 10.9 | 4.7 | 3.1 | 1.38 | 7.0 |
| 2 | 11.4 | 4.9 | 3.2 | 1.5 | 7.4 |
| 3 | 11.4 | 4.7 | 3.2 | 1.6 | 6.5 |
| 4 | 10.02 | 4.4 | 1.7 | 2.7 | 7.0 |
| 5 | 9.9 | 4.4 | 1.8 | 2.7 | 7.0 |
| 6 | 11.9 | 4.9 | 1.7 | 3.1 | 7.4 |
| 7 | 11.0 | 5.0 | 2.0 | 3.0 | 6.3 |
| 8 | 10.2 | 4.5 | 1.8 | 2.8 | 6.5 |
| 9 | 10.7 | 4.6 | 1.8 | 2.9 | 7.4 |
| 10 | 10.8 | 4.6 | 1.9 | 3.0 | 7.0 |
| 11 | 9.7 | 4.6 | 2.1 | 2.6 | 7.4 |
| 12 | 9.3 | 4.6 | 2.5 | 2.6 | 6.5 |
| 13 | 9.7 | 4.5 | 2.6 | 2.7 | 6.3 |
| 14 | 13.8 | 5.3 | 2.5 | 3.0 | 6.8 |
| 15 | 11.5 | 4.5 | 2.6 | 2.9 | 7.1 |

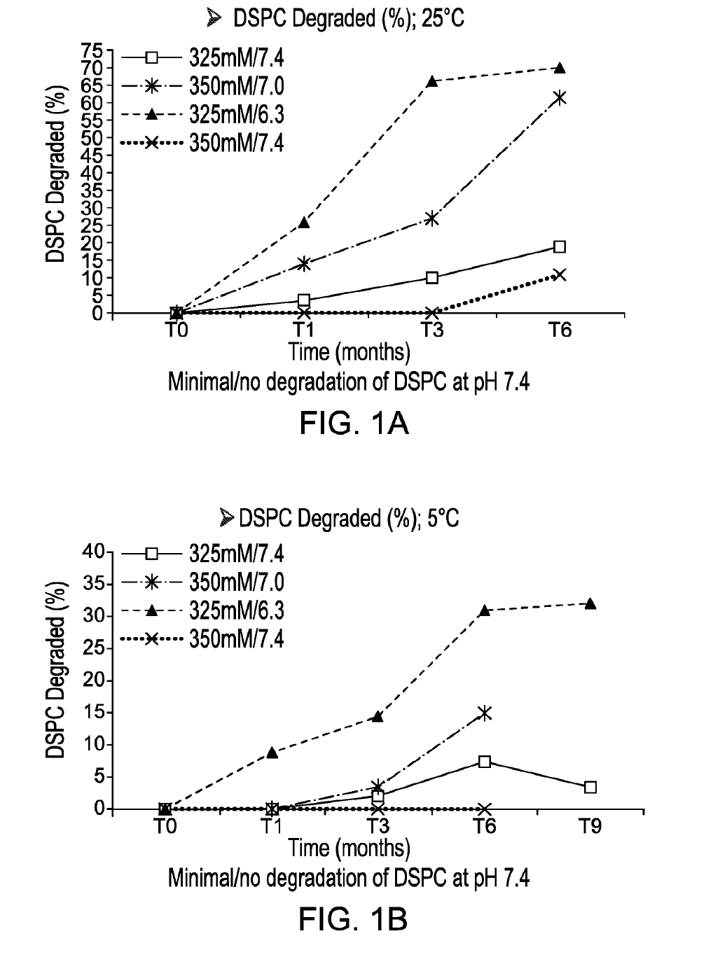

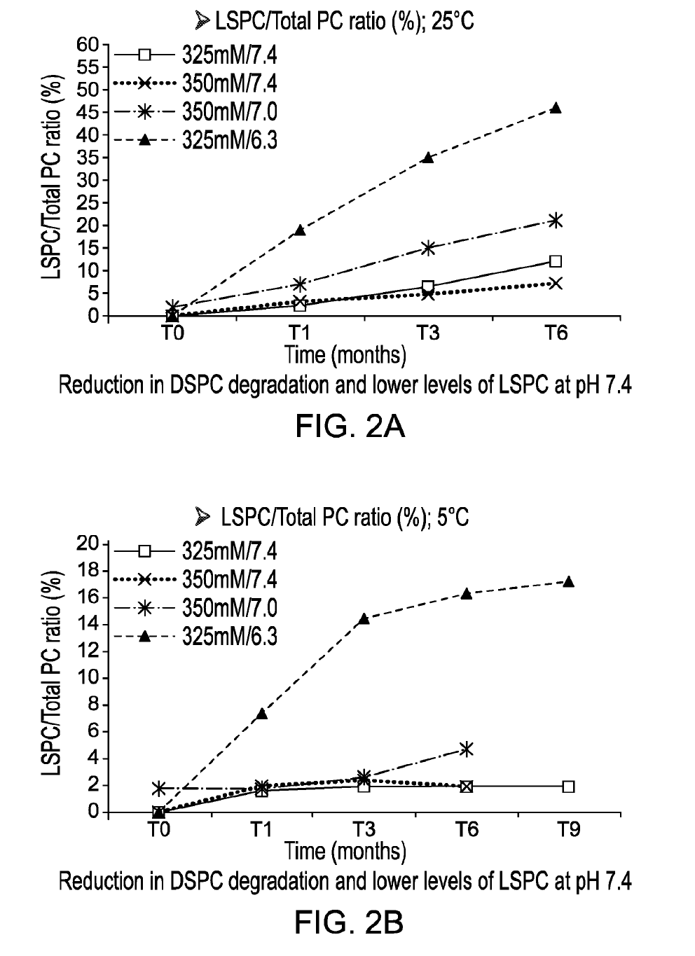

As shown in FIG. 1, the percent DSPC hydrolysis is dependent on the temperature and the pH. As expected, the percent DSPC hydrolysis increased with increasing temperature. Surprisingly, however, the percent DSPC degradation significantly decreased from pH 6.3 to 7.4. Further, there was a 4-fold decrease in the percent DSPC degradation from pH 7.0 to 7.4. This was unexpected as the prior art teaches that the optimal pH for liposomal formulations is 6.5, and that above and below this pH, lipid hydrolysis increases (see, e.g., Grit et al., Hydrolysis of phosphatidylcholine in aqueous liposome dispersions, International Journal of Pharmaceutics, 50(1):1-89 (1989); Grit et al., Chemical stability of liposomes: implications for their physical stability, Chem Phys Lipids., 64:3-18 (1993); and Mrafzali et al., Application of liposomes in food industry, Microencapsulation in the Food Industry, A Practical Implementation Guide edited by Anilkumar G. Gaonkar, Niraj Vasisht, Atul R. Khare, Robert Sobel, pp. 142-144, (2014)). Similarly, FIG. 2 shows the percent ratio of LSPC to total phosphatidylcholine (PC) lipid decreased from pH 6.3 to 7.4. This decrease in the percent ratio of LSPC to total PC is consistent with the decrease in DSPC degradation from pH 6.3 to 7.4.

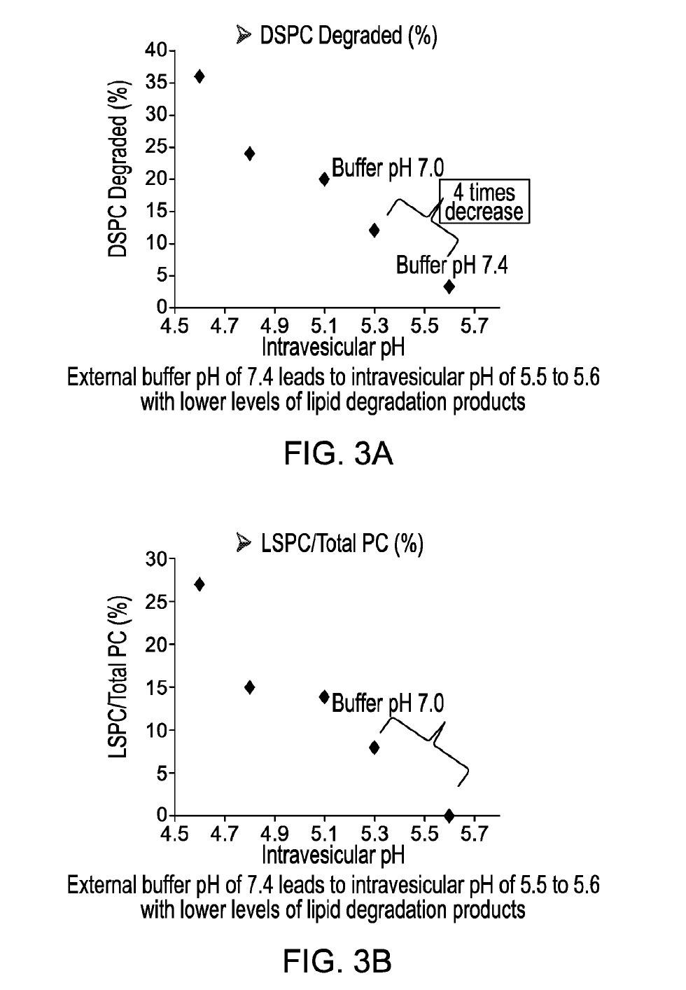

The effect of intravesicular pH on stability of the liposomal formulations was also evaluated. As shown in FIG. 3, intravesicular pH plays an important role in lipid degradation kinetics. An intravesicular pH between about 5.5 to 5.8 yielded the lowest DSPC degradation and LSPC/total PC ratio.

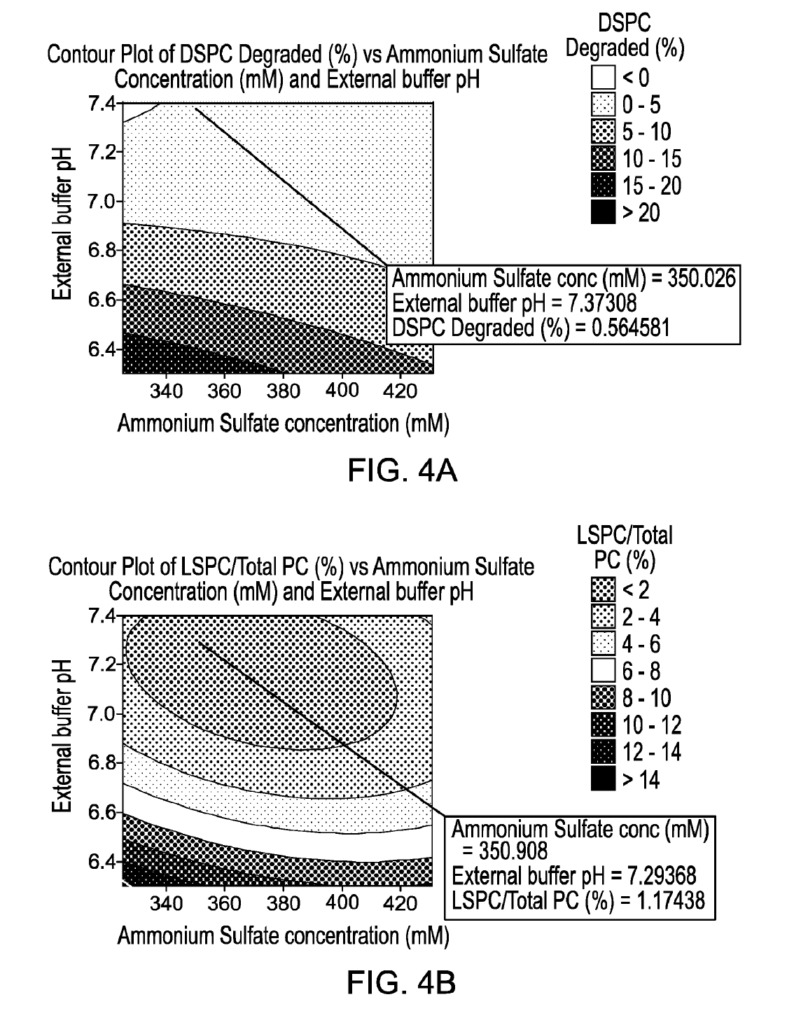

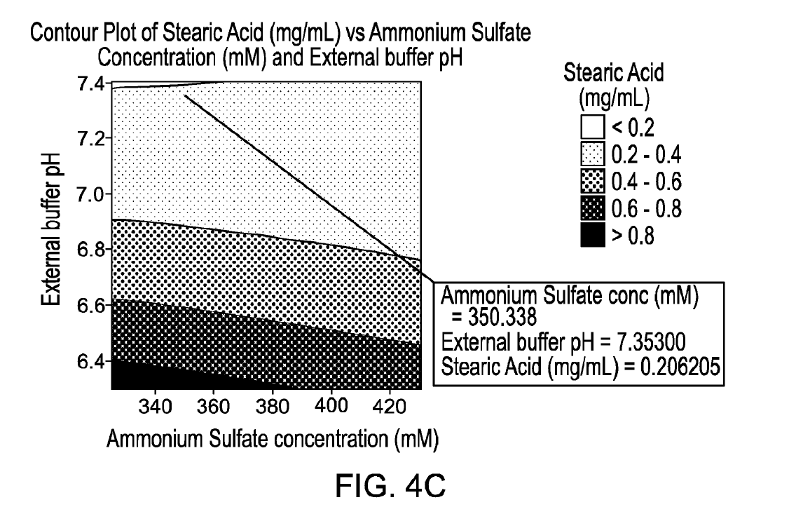

As shown in FIG. 4, the concentration of ammonium sulfate also plays a role in the lipid hydrolysis in the liposomal formulations. FIG. 4A shows a contour plot of the percent degradation of DSPC with ammonium sulfate concentration and external pH. FIG. 4B shows a contour plot of the percent ratio of LSPC/total PC with ammonium sulfate concentration and external pH. FIG. 4C shows a contour plot of the concentration (mg/mL) of stearic acid with ammonium sulfate concentration and external pH. All the contour plot show that an ammonium sulfate concentration (mM) of about 325 mM to about 350 mM minimizes the DSPC hydrolysis and formation of LSPC and stearic acid.

In sum, these results demonstrated that an external pH of about 7.1 to about 7.4, a concentration of ammonium sulfate of 325 to 350 mM, and/or an internal pH of 5.5 to 5.8 minimizes the lipid hydrolysis and formation of LSPC.

What is claimed is:

1. A liposomal formulation comprising:
i) a liposome comprising
a) at least one phosphatidylcholine (PC) lipid;
b) a sterol;
c) a poly(ethylene glycol)-lipid derivative (PEG-lipid); and
d) a therapeutic agent; and
ii) a pharmaceutically-acceptable excipient;
wherein the liposomal formulation has a final pH of about 7.1 to about 7.4.

2. The liposomal formulation of claim 1, wherein the therapeutic agent is selected from the group consisting of amphotericin B, doxorubicin, bupivacaine camptothecin, carfilzomib, daunorubicin, vincristine, cisplatin, oxaloplatin, irinotecan, topotecan, annamycin, cytarabine, paclitaxel, sunitinib, imatinib, lapatinib, mitomycin C, epirubicin, pirarubicin, rubidomycin, carcinomycin, N-acetyladriamycin, rubidazone, 5-imido daunomycin, N-acetyl daunomycin, daunory line, mitoxanthrone, morphine, camptothecin, 9-aminocamptothecin, 7-ethylcamptothecin, 7-Ethyl-10-hydroxy-camptothecin, 10-hydroxycamptothecin, 9-nitrocamptothecin, 10, 11-methylenedioxycamptothecin, 9-amino-10, 11-methylenedioxycamptothecin, 9-chloro-10, 11-methylenedioxycamptothecin, irinotecan, lurtotecan, silatecan, (7-(4-methylpiperazinomethylene)-10,11-ethylenedioxy-20(S)-camptothecin, 7-(4-methylpiperazinomethylene)-10, II-methylenedioxy-20(S)-camptothecin, 7-(2-N-isopropylamino)ethyl)-(20S)-camptothecin, CKD-602, vincristine, vinblastine, vinorelbine, vinflunine, vinpocetine, vindesine, verteporfin, ellipticine, 6-3-aminopropyl-ellipticine, 2-diethylaminoethyl-ellipticinium, datelliptium, retelliptine, paclitaxel, docetaxel, diclofenac, bupivacaine, 17-Dimethylaminoethylamino-17-demethoxygeldanamycin, cetirizine, fexofenadine, primidone and other catecholamines, epinephrine, (S)-2-(2,4-dihydroxyphenyl)-4,5-dihydro-4-methyl-4-thiazolecarboxylic acid (deferitrin), (S)-4,5-dihydro-2-(3-hydroxy-2-pyridinyl)-4-methyl-4-thiazolecarboxylic acid (desferrithiocin), (S)-4,5-dihydro-2-[2-hydroxy-4-(3,6,9,12-tetraoxamidecyloxy)phenyl]-4-methyl-4-thiazolecarboxylic acid, (S)-4,5-dihydro-2-[2-hydroxy-4-(3,6-dioxaheptyloxy)phenyl]-4-methyl-4-thiazolecarboxylic acid, ethyl (S)-4,5-dihydro-2-[2-hydroxy-4-(3,6-dioxaheptyloxy)phenyl]-4-methyl-4-thiazolecarboxylate, (S)-4,5-dihydro-2-[2-hydroxy-3-(3,6,9-trioxadecyloxy)]-4-methyl-4-thiazolecarboxylic acid and desazadesferrithiocin, including salts, prodrugs and derivatives thereof.

3. The liposomal formulation of claim 1, wherein the therapeutic agent is a docetaxel or a derivative thereof esterified at the 2′-O-position with a heterocyclyl-(C₂₋₅alkanoic acid).

4. The liposomal formulation of claim 1, wherein the final pH is about 7.3.

5. The liposomal formulation of claim 1, wherein the liposome has an intravesicular pH of about 5.5 to about 5.8.

6. The liposomal formulation of claim 1, wherein the phosphatidylcholine (PC) lipid is:
i) a saturated phosphatidylcholine lipid selected from the group consisting of: 1,2-dilauroyl-sn-glycero-3-phosphocholine (DLPC), 1,2-dimyristoyl-sn-glycero-3-phosphocholine (dimyristoylphosphatidylcholine; DMPC), 1,2-distearoyl-sn-glycero-3-phosphocholine (distearoylphosphatidylcholine; DSPC), 1,2-dioleoyl-sn-glycero-3-phosphocholine (DOPC), 1,2-dipalmitoyl-sn-glycero-3-phosphocholine (dipalmitoylphosphatidylcholine; DPPC), 1-myristoyl-2-palmitoyl-sn-glycero-3-phosphocholine (MPPC), 1-palmitoyl-2-myristoyl-sn-glycero-3-phosphocholine (PMPC), 1-myristoyl-2-stearoyl-sn-glycero-3-phosphocholine (MSPC), 1-palmitoyl-2-stearoyl-sn-glycero-3-phosphocholine (PSPC), 1-stearoyl-2-palmitoyl-sn-glycero-3-phosphocholine (SPPC) and 1-stearoyl-2-myristoyl-sn-glycero-3-phosphocholine (SMPC); or
ii) a unsaturated phosphatidylcholine lipid selected from the group consisting of: 1,2-dimyristoleoyl-sn-glycero-3-phosphocholine, 1,2-dimyristelaidoyl-sn-glycero-3-phosphocholine, 1,2-dipamiltoleoyl-sn-glycero-3-phosphocholine, 1,2-dipalmitelaidoyl-sn-glycero-3-phosphocholine, 1,2-dioleoyl-sn-glycero-3-phosphocholine (DOPC), 1,2-dielaidoyl-sn-glycero-3-phosphocholine, 1,2-dipetroselenoyl-sn-glycero-3-phosphocholine, 1,2-dilinoleoyl-sn-glycero-3-phosphocholine, 1-palmitoyl-2-oleoyl-sn-glycero-3-phosphocholine (palmitoyloleoylphosphatidylcholine; POPC), 1-palmitoyl-2-linoleoyl-sn-glycero-3-phosphocholine, 1-stearoyl-2-oleoyl-sn-glycero-3-phosphocholine (SOPC), 1-stearoyl-2-linoleoyl-sn-glycero-3-phosphocholine, 1-oleoyl-2-myristoyl-sn-glycero-3-phosphocholine (OMPC), 1-oleoyl-2-palmitoyl-sn-glycero-3-phosphocholine (OPPC) and 1-oleoyl-2-stearoyl-sn-glycero-3-phosphocholine (DSPC).

7. The liposomal formulation of claim 1, wherein the phosphatidylcholine lipid is selected from the group consisting of: DPPC, DSPC, hydrogenated soy PC (HSPC) and mixtures thereof.

8. The liposomal formulation of claim 1, wherein the sterol is cholesterol.

9. The liposomal formulation of claim 1, wherein the PEG-lipid is selected from the group consisting of: diacyl-phosphatidylethanolamine-N-[methoxy(polyethene glycol)]; distearoyl-phosphatidylethanolamine-N-[methoxy(polyethylene glycol)-2000] (DSPE-PEG-2000); and distearoyl-phosphatidylethanolamine-N-[methoxy(polyethylene glycol)-5000] (DSPE-PEG-5000).

10. The liposomal formulation of claim 9, wherein the PEG-lipid is DSPE-PEG-2000.

11. The liposomal formulation of claim 1, wherein the liposome contains:
i) about 45 mol % to about 70 mol % phosphatidylcholine;
ii) about 30 mol % to about 50 mol % cholesterol; and
iii) about 1 mol % to about 10 mol % PEG-lipid.

12. The liposomal formulation of claim 3, wherein:
i) the ratio of the combined weight of the phosphatidylcholine and the sterol to the weight of the docetaxel or its derivative is about 1:0.01 to about 1:1; and
ii) the molar ratio of the combined phosphatidylcholine and sterol to the PEG-lipid is about 1000:1 to about 20:1.

13. The liposomal formulation of claim 12, wherein:
i) the ratio of the combined weight of the phosphatidylcholine and the sterol to the weight of the docetaxel derivative is about 1:0.2, and
ii) the molar ratio of the combined phosphatidylcholine and sterol to the PEG-lipid is about 27:1.

14. The liposomal formulation of claim 1, wherein the liposomal formulation comprises:
i) from about 50 mol % to about 70 mol % of DSPC and from about 25 mol % to about 45 mol % of cholesterol,
ii) about 53 mol % of DSPC, about 44 mol % of cholesterol and about 3 mol % of DSPE-PEG-2000, or
iii) about 66 mol % of DSPC, about 30 mol % of cholesterol and about 4 mol % of DSPE-PEG-2000.

15. The liposomal formulation of claim 3, wherein the docetaxel derivative has the following formula: 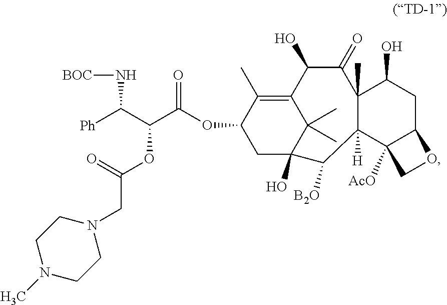
or a pharmaceutically-acceptable salt of TD-1 thereof.

16. The liposomal formulation of claim 1 further comprising at least a diagnostic agent or a targeting agent.

17. A pharmaceutical composition comprising the liposomal formulation of claim 1 for treating cancer in a patient in need thereof, wherein the pharmaceutical composition is suitable for parenteral administration.

18. The pharmaceutical composition of claim 17, wherein the cancer is:
i) a solid tumor cancer selected from the group consisting of bladder cancer, breast cancer, cervical cancer, colorectal cancer (CRC), esophageal cancer, gastric cancer, head and neck cancer, hepatocellular cancer, lung cancer, melanoma, neuroendocrine cancer, ovarian cancer, pancreatic cancer, prostate cancer, renal cancer and thymus cancer;
ii) a hematological malignancies selected from the group consisting of multiple myeloma, T-cell lymphoma, B-cell lymphoma, Hodgkin's disease, non-Hodgkins lymphoma, acute myeloid leukemia and chronic myelogenous leukemia; or
iii) an epithelial cancer.

19. A method for improving the stability of a liposomal formulation, the method comprising:
i) providing a liposomal formulation comprising:
a) a liposome comprising:
1) at least one phosphatidylcholine lipid;
2) a sterol;
3) a poly(ethylene glycol)-lipid derivative (PEG-lipid); and
4) a docetaxel or a derivative thereof esterified at the 2′-O-position with a heterocyclyl-(C₂₋₅alkanoic acid); and
b) a pharmaceutically acceptable excipient; and
ii) adjusting the pH of the liposomal formulation to about 7.1 to about 7.4.

20. The method of claim 19, further comprising the step of:
iii) maintaining an intravesicular pH of the liposome between about 5.5 to about 5.8.

21. The method for improving stability of the liposomal formulation of claim 20, wherein the final pH is about 7.3.

22. A method for preparing a stable liposomal formulation comprising the steps of:
i) forming a first liposome having a lipid bilayer including a phosphatidylcholine lipid and a sterol, wherein the lipid bilayer encapsulates an interior environment containing an aqueous solution;
ii) loading the first liposome with a therapeutic agent, or a pharmaceutically-acceptable salt thereof, to form a loaded liposome;
iii) incorporating a PEG-lipid into the lipid bilayer of the loaded liposome; and
iv) adjusting the pH of the liposomal formulation to about 7.1 to about 7.4.

23. The method according to claim 22, wherein the pH is adjusted to about 7.3.

24. The method according to claim 22, wherein the therapeutic agent is docetaxel esterified at the 2′-O-position with a heterocyclyl-(C₂₋₅alkanoyl) group.

25. The method of claim 22, wherein
a) the loading of the first liposome employs an ion-gradient comprising an ammonium sulfate buffer at concentrations between about 325 mM to about 350 mM, and
b) the liposome has an intravesicular pH between about 5.5 to about 5.8.
